(19) United States
(12) Patent Application Publication
(10) Pub. No.: US 2024/0239949 A1
(43) Pub. Date: Jul. 18, 2024

(54) MULTIFUNCTIONAL EPOXY COMPOUND HAVING MULTIPLE LIQUID CRYSTAL CORES, AND CURED PRODUCT PRODUCED THEREFROM

(71) Applicant: KYUNGPOOK NATIONAL UNIVERSITY INDUSTRY-ACADEMIC COOPERATION FOUNDATION, Daegu (KR)

(72) Inventors: Hyeon Uk YEO, Daegu (KR); Thi en TRINH, Daegu (KR); Kyo Sun KU, Daegu (KR)

(73) Assignee: KYUNGPOOK NATIONAL UNIVERSITY INDUSTRY-ACADEMIC COOPERATION FOUNDATION, Daegu (KR)

(21) Appl. No.: 18/556,977

(22) PCT Filed: Apr. 12, 2022
(86) PCT No.: PCT/KR2022/005273

§ 371 Date: Oct. 24, 2023

(30) Foreign Application Priority Data
Apr. 27, 2021 (KR) 10-2021-0054162

(51) Int. Cl. C08G 59/22; C07D 303/18; C07D 407/12; C08G 59/50; C08L 63/00

(52) U.S. Cl. CPC C08G 59/22; C07D 303/18; C07D 407/12; C08G 59/5033; C08L 63/00

(57) ABSTRACT

There are provided a multifunctional epoxy compound obtained by modifying two or more epoxide functional groups on terminal portions of liquid crystal molecules synthesized by bonding multiple thermotropic liquid crystal materials; and a cured product produced by reacting the compound with a curing agent. The compound and the cured product enable easy interaction between liquid crystals and exhibiting high thermal conductivity. They are usable alone as a heat-dissipating polymer, or in the form of a composite material, and are applicable as a matrix resin of a composite material.

### CROSS-REFERENCE TO PRIOR APPLICATIONS

This Application is a National Stage Patent Application of PCT International Application No. PCT/KR2022/005273 (filed on Apr. 12, 2022), which claims priority to Korean Patent Application No. 10-2021-0054162 (filed on Apr. 27, 2021), which are all hereby incorporated by reference in their entirety.

### BACKGROUND

The present invention relates to a multifunctional epoxy compound obtained by modifying two or more epoxide functional groups on terminal portions of liquid crystal molecules synthesized by bonding multiple thermotropic liquid crystal materials; and a cured product produced by reacting the compound with a curing agent. More specifically, the present invention relates to a compound and a cured product enabling easy interaction between liquid crystals and exhibiting high thermal conductivity, thereby being usable alone as a heat-dissipating polymer, or in the form of a composite material, and being applicable as a matrix resin of a composite material.

An epoxy resin is a thermosetting resin composed of a network polymer formed by ring opening of an epoxy group that is generated when an epoxy compound having two or more epoxy groups in a molecule is mixed with a curing agent. An epoxy resin has excellent resistance to chemical components, durability, and low volumetric shrinkage during curing so that it is used as an essential high-functionality raw material in all industrial fields such as adhesives, paints, electronics/electricity, and civil engineering/architecture.

Currently, the most widely used method to increase the thermal conductivity of an epoxy resin is to create a composite material by mixing thermally conductive fillers such aluminum oxide and aluminum nitride. Composite materials manufactured in this manner have the advantages of existing epoxy resins while exhibiting relatively high thermal conductivity. However, due to the nature of fillers, which have physical properties that are fixed to some extent, it is difficult to make changes beyond simply increasing the filler content in order to manufacture a composite material with higher thermal conductivity. In this case, an excessively high filler content may make manufacturing difficult or there is a possibility that unexpected disadvantages may arise in other physical properties. Hence, research has been conducted to manufacture composite materials with improved thermal conductivity by increasing the inherent thermal conductivity of not only a filler but also an epoxy resin without increasing the filler content.

The widely used epoxy resin in the form of diglydicyl ether of bisphenol A (DGEBA) is known to form a three-dimensional network cross-linked structure after curing. This three-dimensional structure has the advantage of giving excellent durability and corrosion resistance to a material, but it is disadvantageous in terms of thermal conductivity because the scattering of phonons, which are known to play the role of heat transfer within a polymer, occurs predominantly. In fact, a cured DGEBA epoxy resin is known to have a low thermal conductivity of about 0.2 W/m·K. Therefore, liquid crystalline epoxy resin is being studied to change this three-dimensional structure into a more aligned one-dimensional or two-dimensional structure.

Meanwhile, research has also been conducted to improve the physical properties of compounds and cured resins by introducing flexible groups into these liquid crystalline epoxy compounds. When a flexible group is introduced, the solubility of a compound increases, thereby making it easier to handle in a process, the interaction between mesogens may be adjusted to change the temperature range where liquid crystallinity appears, the arrangement form in a liquid crystal state may also be changed, and the curing speed may be controlled by adjusting the reactivity of an epoxy group.

However, most of these compounds are in a form where mesogens are present inside the compounds and epoxy groups are present at both ends thereof. In this case, improvement of thermal conductivity, which is desired through the introduction of a liquid crystalline epoxy compound, may be inefficient.

### SUMMARY

An object of the present invention is to synthesize liquid crystal molecules by combining thermotropic liquid crystal materials through chemical modification and to provide a multifunctional epoxy compound in which two or more epoxide functional groups are modified at the terminal portions thereof.

Another object of the present invention is to provide a cured product produced by reacting the multifunctional epoxy compound, which has high thermal conductivity and thus may be used as a heat dissipating polymer and a composite material, with a curing agent.

The technical problems to be achieved by the invention are not limited to the technical problems mentioned above, and other technical problems that are not mentioned may be clearly understood by those skilled in the art from the description of the present invention.

To achieve the objects described above, the present invention provides a multifunctional epoxy compound and a cured product produced therefrom.

The present invention provides a multifunctional epoxy compound represented by Formula (I) below:

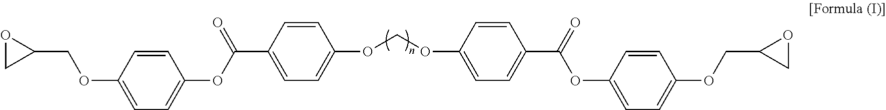

[In the Formula, n is an Integer in a Range from 1 to 30.]

The present invention provides a multifunctional epoxy compound represented by Formula (II) below:

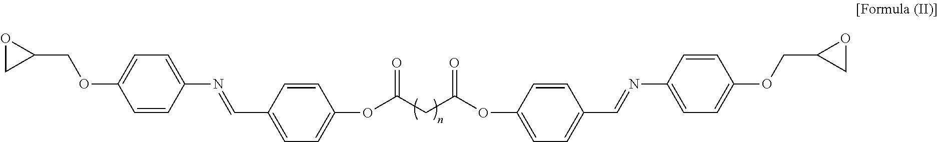

[In the Formula, n is an Integer in a Range from 1 to 30.]

The present invention provides a multifunctional epoxy compound represented by Formula (III) below:

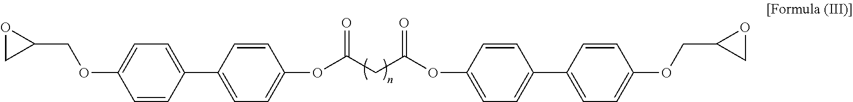

[In the Formula, n is an Integer in a Range from 1 to 30.]

The present invention provides a cured epoxy resin product obtained by reacting a multifunctional epoxy compound represented by Formula (I) above with a curing agent.

The present invention provides a cured epoxy resin product obtained by reacting a multifunctional epoxy compound represented by Formula (II) above with a curing agent.

The present invention provides a cured epoxy resin product obtained by reacting a multifunctional epoxy compound represented by Formula (III) above with a curing agent.

The present invention provides a cured epoxy resin product represented by Formula (IV) below:

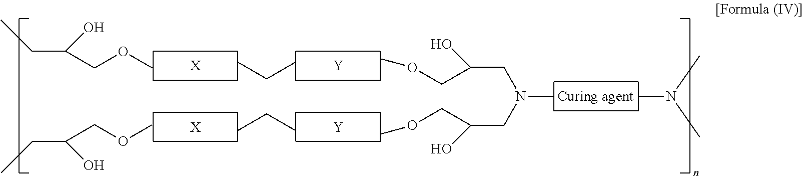

[In the Formula, X and Y May be the Same or Different, and are Selected from the Compounds Represented by Formulas (I) to (III).]

The curing agent may be selected from the group consisting of 4,4′-diaminodiphenylmethane (DDM), diaminodiphenylsulfone (DDS), m-phenylenediamine (mPDA), and dicyandiamide (DICY).

The cured epoxy resin product can be used in substrates, compounds, adhesives, pads, heat spreads, and heat sinks.

All matters mentioned above about the multifunctional epoxy compounds and the cured epoxy resins prepared therefrom apply equally, unless contradictory.

The novel epoxy resin and the cured product thereof according to the present invention facilitate interactions between liquid crystals and improve thermal conductivity so that they can be used alone as a heat dissipating polymer or in the form of a composite material.

The effects of the present invention are not limited to the effects mentioned above, and other effects that are not mentioned will be clearly understood by those skilled in the art from the recitations of the claims.

### BRIEF DESCRIPTION OF THE DRAWINGS

FIG. 1 is a polarizing microscope image of Example 1 ((a), (b): EBH-4, (c), (d): EBH-6, (e), (f): EBH-8)) of the present invention.

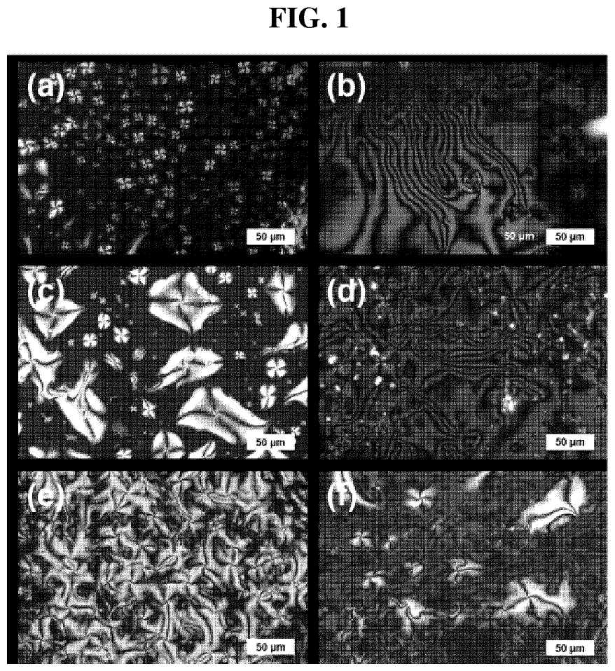

FIG. 2 is a polarizing microscope image of Example 2 ((a), (b): EIM-4, (c), (d): EIM-6, (e), (f): EIM-8)) of the present invention.

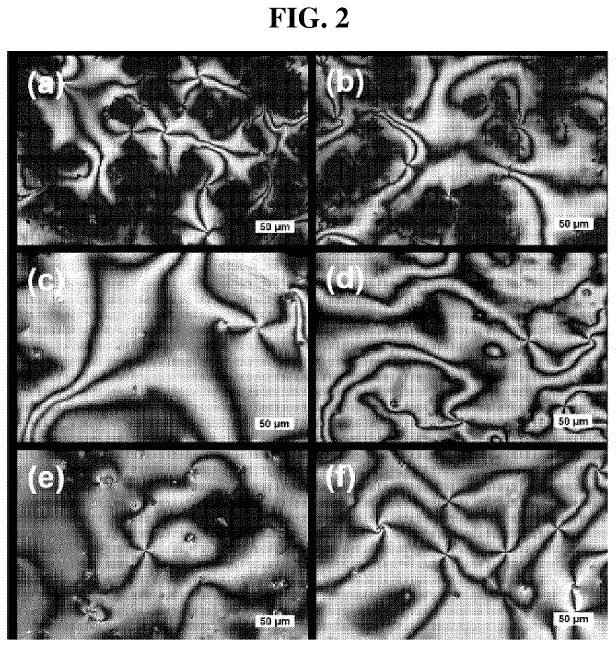

FIG. 3 is a polarizing microscope image of Example 3 ((a): EBP-4, (b): EBP-6, (c): EBP-7, (d): EBP-8) of the present invention.

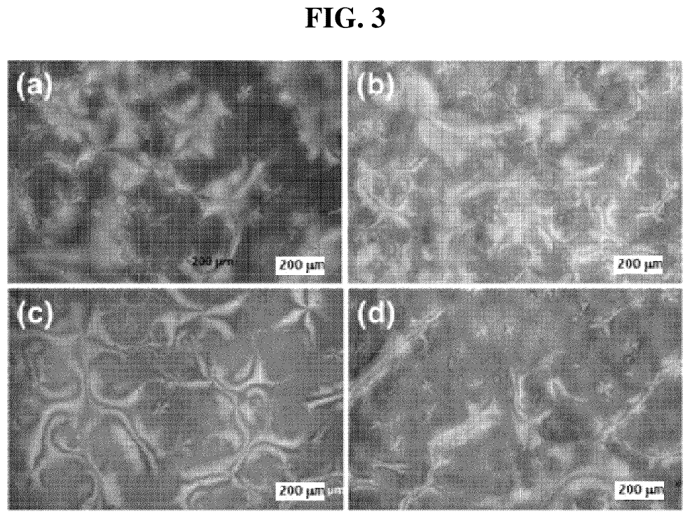

### DETAILED DESCRIPTION

The terms used in this specification were selected as general terms that are currently widely used as much as possible while considering the function in the present invention, but they may vary depending on the intention of one of ordinary skill in the art, or a precedent or the emergence of new technology, etc. In addition, in certain cases, there are terms arbitrarily selected by the applicant, and in this case, the meaning will be described in detail in the corresponding description of the invention. Therefore, the terms used in the present invention should be defined based on the meaning of the terms and the overall content of the present invention, rather than simply the name of the terms.

Unless otherwise defined, all terms used herein, including technical or scientific terms, have the same meaning as commonly understood by one of ordinary skill in the technical field to which the present invention pertains. Terms defined in commonly used dictionaries should be interpreted as having meanings consistent with the meanings they have in the context of the related technology, and unless clearly defined in the present application, they should not be interpreted in an idealized or excessively formal sense.

The numerical range includes the values defined in the range above. Any maximum numerical limit given throughout this specification includes all lower numerical limits as if the lower numerical limit were clearly written. Every minimum numerical limit given throughout this specification includes every higher numerical limit as if such higher numerical limit were clearly written. All numerical limits given throughout this specification will include all better numerical ranges within a broader numerical range, as if narrower numerical limits were clearly written.

The present invention provides a multifunctional epoxy compound represented by Formula (I) below:

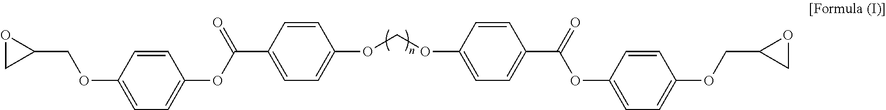

[In the Formula, n is an Integer in a Range from 1 to 30.]

The compound of Formula (I) above may be prepared using bis (4-hydroxyphenyl) 4,4′-(alkane-1, n-diylbis (oxy)) dibenzoate as a starting material.

The preparation of the compound of Formula (I) above may be performed under basic conditions, for example, in the presence of sodium hydroxide.

The reaction for preparing the compound of Formula (I) above may be carried out at a temperature in a range from 80 to 105° C., and preferably at a temperature in a range from 85 to 95° C.

The preparation time for the compound of Formula (I) above may be in a range from 0.5 hour to 4 hours, preferably from 1 hour to 2 hours.

The preparation n of the compound of Formula (I) above may be performed in the presence of water or an alcohol, and the type of alcohol is not limited as long as it is known in the art.

The present invention provides a cured epoxy resin product obtained by reacting a multifunctional epoxy compound represented by Formula (I) above with a curing agent.

The curing agent may be selected from the group consisting of 4,4′-diaminodiphenylmethane (DDM), diaminodiphenylsulfone (DDS), m-phenylenediamine (mPDA), and dicyandiamide (DICY).

The cured epoxy resin product can be used in the fields of substrates, compounds, adhesives, pads, heat spreads, and heat sinks.

The molar mixing ratio of the compound and the curing agent may be 1.5:1 to 4:1, preferably 1.5:1 to 2:1.

The production of the cured product may be performed by using methods such as hot press molding, injection molding, and roll molding, but this is not particularly limited.

The temperature for producing the cured product may be in a range from 100 to 150° C., preferably in a range from 120 to 150° C.

The preparation time of the cured product may be 0.5 to 1.5 hours, preferably 0.5 to 1 hour.

The glass transition temperature of the cured product may be measured by using a differential scanning calorimeter, and may be in a range from 79 to 127° C.

The glass transition temperature may be inversely proportional to a chain length.

The present invention provides a multifunctional epoxy compound represented by Formula (II) below:

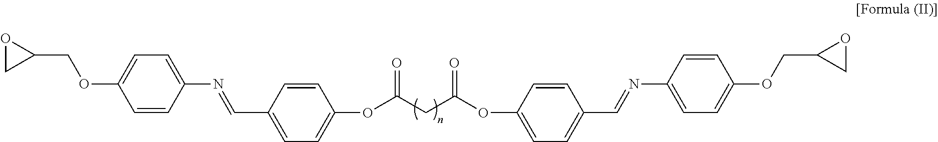

[In the Formula, n is an Integer in a Range from 1 to 30.]

The compound of Formula (II) above may be prepared from bis (4-(((4-hydroxyphenyl) imino) methyl) phenyl) alkanedioate prepared by using bis (4-formylphenyl) alkanedioate as a starting material.

The bis (4-(((4-hydroxyphenyl) imino) methyl) phenyl) alkanedioate may be prepared in the presence of an alcohol, and the alcohol may be selected from the group consisting of methanol, ethanol, propanol, butanol, pentanol, hexanol, and heptanol.

The bis (4-(((4-hydroxyphenyl) imino) methyl) phenyl) alkanedioate may be prepared at a temperature in a range from 50 to 80° C., and may be produced preferably at a temperature in a range from 65 to 75° C.

The preparation of the compound of Formula (II) above may be performed in the presence of a solvent such as dimethylformamide (DMF), N-methylpyrilidone (NMP), N, N′-dimethylacetamide (DMAc), dimethylsulfuroxide (DMSO), and tetrahydrofuran (THE), metacresol (m-cresol) or a mixture thereof.

The preparation of the compound of Formula (II) above may be performed at a temperature in a range from 75 to 100° C., preferably at a temperature in a range from 85 to 95° C., and may be performed for 0.5 to 1.5 hours, preferably, may be performed for 0.5 to 1 hour.

The compound of Formula (II) above may be washed with an alcohol selected from the group consisting of methanol, ethanol, propanol, butanol, pentanol, hexanol, and heptanol.

The present invention provides a cured epoxy resin product obtained by reacting a multifunctional epoxy compound represented by Formula (II) above with a curing agent.

The curing agent may be selected from the group consisting of 4,4′-diaminodiphenylmethane (DDM), diaminodiphenylsulfone (DDS), m-phenylenediamine (mPDA), and dicyandiamide (DICY).

The cured epoxy resin product can be used in the fields of substrates, compounds, adhesives, pads, heat spreads, and heat sinks.

The molar mixing ratio of the compound and the curing agent may be 1.5:1 to 4:1, preferably 1.5:1 to 2:1.

The production of the cured product may be performed by using methods such as hot press molding, injection molding, and roll molding, but this is not particularly limited.

The temperature for producing the cured product may be in a range from 100 to 150° C., preferably in a range from 120 to 150° C.

The preparation time of the cured product may be 0.5 to 1.5 hours, preferably 0.5 to 1 hour.

The glass transition temperature of the cured product may be measured by using a differential scanning calorimeter, and may be in a range from 90 to 123° C.

The glass transition temperature may be inversely proportional to a chain length.

The present invention provides a multifunctional epoxy compound represented by Formula (III) below:

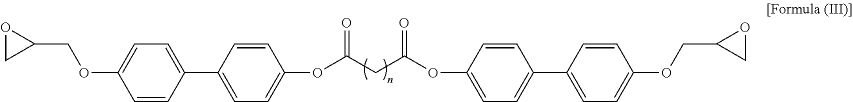

[In the Formula, n is an Integer in a Range from 1 to 30.]

The compound of Formula (III) above may be prepared by using bis (4′-(allyloxy)-[1,1′-biphenyl]-4-yl) alkanedioate as a starting material.

The preparation of the compound of Formula (III) above may be performed at a temperature in a range from 50 to 80° C., and preferably at a temperature in a range from 55 to 65° C.

The preparation of the compound of Formula (III) above may be performed in the presence of chloroform.

The preparation of the compound of Formula (III) above may be performed in the presence of a solvent selected from the group consisting of acetone, chloroform, ethanol, methanol, and methylene chloride.

The present invention provides a cured epoxy resin product obtained by reacting a multifunctional epoxy compound represented by Formula (III) above with a curing agent.

The curing agent may be selected from the group consisting of 4,4′-diaminodiphenylmethane (DDM), diaminodiphenylsulfone (DDS), m-phenylenediamine (mPDA), and dicyandiamide (DICY).

The cured epoxy resin product can be used in the fields of substrates, compounds, adhesives, pads, heat spreads, and heat sinks.

The molar mixing ratio of the compound and the curing agent may be 1.5:1 to 4:1, preferably 1.5:1 to 2:1.

Production of the cured product may be performed by using methods such as hot press molding, injection molding, and roll molding, but this is not particularly limited.

The temperature for producing the cured product may be in a range from 160 to 200° C., preferably in a range from 180 to 200° C.

The preparation time of the cured product may be 1 to 3.5 hours, preferably 1.5 to 2.5 hour.

The glass transition temperature of the cured product may be measured by using a differential scanning calorimeter, and may be in a range from 125 to 150° C.

In addition, the cured product may be a cured epoxy resin product represented by Formula (IV) below:

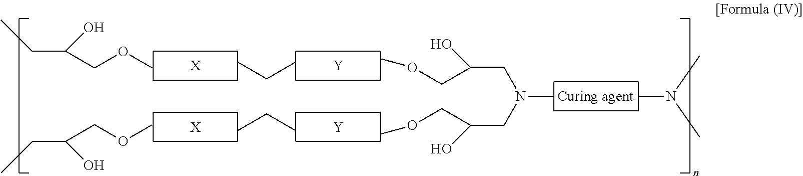

[In the Formula, X and Y May be the Same or Different, and are Selected from the Compounds Represented by Formulas (I) to (III).]

The curing agent may be selected from the group consisting of 4,4′-diaminodiphenylmethane (DDM), diaminodiphenylsulfone (DDS), m-phenylenediamine (mPDA), and dicyandiamide (DICY).

Hereinafter, examples of the present invention will be described in detail, but it is obvious that the present invention is not limited to the examples described below.

The advantages and features of the present invention and methods for achieving them will become clear with reference to the examples described in detail below. However, the present invention is not limited to the examples disclosed below and may be implemented in various different forms, and the examples are only provided to ensure that the disclosure of the present invention is complete and to fully inform the scope of the invention to those of ordinary skills in the technical field to which the present invention pertains, and the present invention is only defined by the scope of the claims.

### Example 1: Synthesis of EBA-n (bis (4-(oxiran-2-ylmethoxy) phenyl) 4,4′-(alkane-1, n-diylbis (oxy)) dibenzoate)

Scheme 1 below shows the synthesis process of EBH-n.

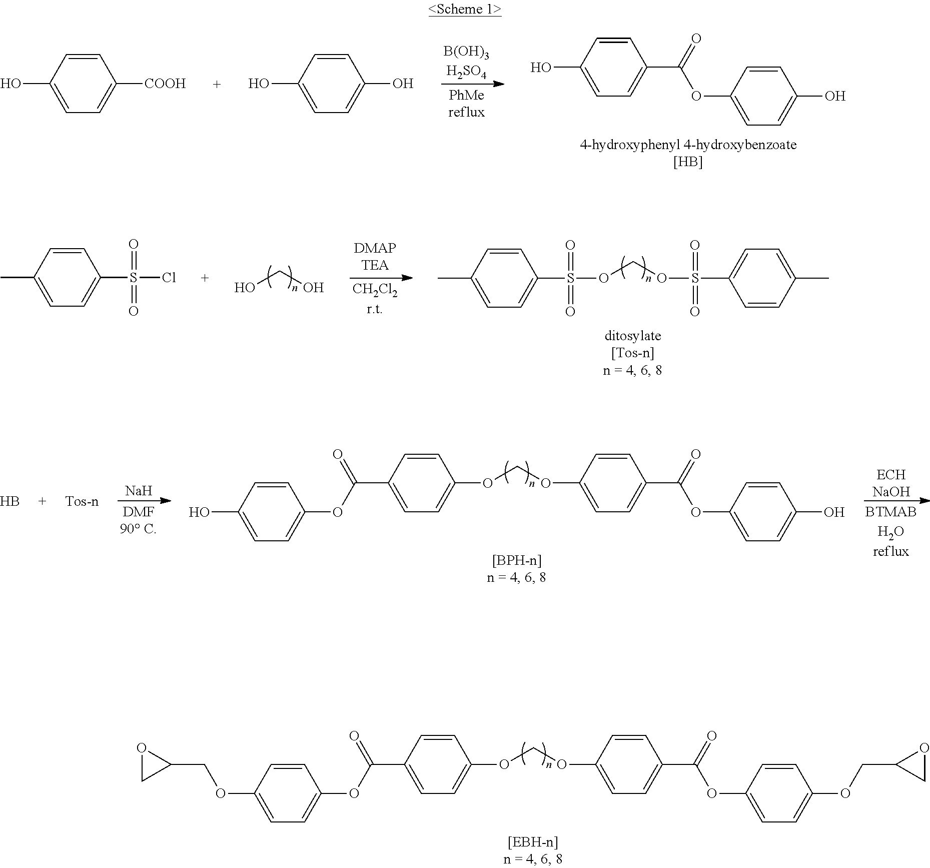

### \textcircled{1} Synthesis of bis (4-hydroxyphenyl) 4,4′-(alkane-1,n-diylbis (oxy)) dibenzoate (BPEn) of Formula (I) Below

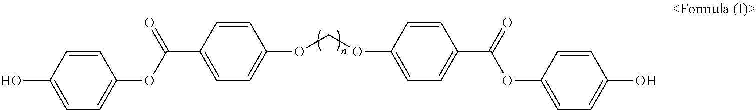

A) n=4

4-hydroxylphenyl-4-hydroxybenzoate (2.00 g, 8.67 mmol) and butane-1,4-diylbis (4-methylbenzenesulfonate) (Tos-4) (1.38 g, 3.47 mmol) 2 were put into a two-necked flask, of which atmosphere was replaced with argon, and then 30 ml of anhydrous DMF and NaH (0.40 g, 4.34 mmol) were added. After stirring at 90° C. for 3 days, the solvent was removed, and the obtained solid material was purified through silica column chromatography using a hexane:ethyl acetate solution in a volume ratio of 7:3 as an eluent. The yield was 67%.

¹H NMR (500 MHZ, DMSO-d₆): δ=9.46 (s, 2H), 8.05 (d, J=9 Hz, 4H), 7.12 (d, J=9 Hz, 4H), 7.04 (d, J=8.5 Hz, 4H), 6.80 (d, J=9 Hz, 4H), 4.07-4.09 (m, 4H), 1.37-1.38 (m, 4H) ppm.

B) n=6

The synthesis was performed in the same manner as A), except that Tos-6 was used instead of Tos-4.

*Tos-n; alkane-1-n-diyl bis (4-methylbenzenesulfonate)

¹H NMR (500 MHZ, DMSO-d₆): δ=8.04 (d, J=9 Hz, 9.46 (s, 2H), 7.10 (d, J=9 Hz, 4H), 7.03 (d, J=9 Hz, 4H), 6.80 (d, J=9 Hz, 4H), 4.11 (t, J=6 Hz, 4H), 1.79-1.80 (m, 4H), 1.50 (s, 4H).

C) n=8

The synthesis was performed in the same manner as A), except that Tos-8 was used instead of Tos-4.

¹H NMR (500 MHZ, DMSO-d₆): δ=9.46 (s, 2H), 8.04 (d, J=9 Hz, 4H), 7.10 (d, J=9 Hz, 4H), 7.02 (d, J=9 Hz, 4H), 6.79 (d, J=9 Hz, 4H), 4.07-4.09 (m, 4H), 1.75-1.743 (m, 4H), 1.41-1.43 (m, 4H), 1.37-1.38 (m, 4H).

### \textcircled{2} Synthesis of bis (4-(oxiran-2-ylmethoxy) phenyl) 4,4′-(alkane-1, n-diylbis (oxy)) dibenzoate (EBH-n) of Formula (II) Below

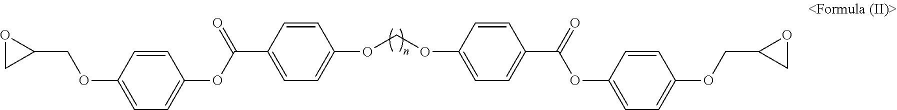

D) n=4

BPH4 (3.00 g, 5.83 mmol) and benzyl trimethylammonium bromide (0.30 g) were placed in a two-necked flask, of which atmosphere was replaced with argon, and then 20 ml of epichlorohydrin was added. Afterwards, sodium hydroxide (0.48 g, 11.66 mmol) was dissolved in 5 ml of distilled water and added to the flask. After a reaction at 90° C. for 1 hour, the residual solvent was removed by using a vacuum rotary evaporator. The obtained solid material was dissolved in acetone and purified twice through a reprecipitation process in distilled water, and EBH-4 in the form of a white solid was obtained with a yield of 97%.

¹H NMR (500 MHZ, CDCl₃): δ=8.14 (d, J=8.5 Hz, 4H), 7.12 (d, J, =9 Hz, 4H), 6.96 (d, J=8.5 Hz, 8H), 4.25 (dd, J=3.5 Hz, 1.5 Hz, 2H), 4.08 (t, J=6.5 Hz, 4H), 3.98 (dd, J=6 Hz, 2 Hz, 2H), 3.37-3.35 (m, 2H), 2.93 (t, J=4.5 Hz, 2H), 2.78 (m, 2H), 2.05 (t, J=3 Hz 4H) ppm. 13C NMR (125 MHZ, CDCl₃): δ=165.19, 163.24, 156.16, 145.01, 132.29, 122.63, 121.93, 115.38, 114.28, 69.28, 67.71, 50.13, 44.72,25.87 ppm. MS (+ESI): calcd for [C₃₆H₃₄O₁₀+H]⁺: m/z 627; found: m/z 627.

E) n=6

The synthesis was performed in the same manner as D), except that BPH6 was used instead of BPH4.

¹H NMR (500 MHZ, CDCl₃): δ=8.13 (d, J=8.5 Hz, 4H), 7.11 (d, J=9 Hz, 4H), 6.96 (d, J=8.5 Hz, 8H), 4.24 (dd, J=3 Hz, 1.5 Hz, 2H), 4.08 (t, J=6.5 Hz, 4H), 3.98 (dd, J=5.5 Hz, 2 Hz, 2H), 3.37 (m, 2H), 2.92 (t, J=4.5 Hz, 2H), 2.77 (m, 2H), 1.89 (t, J=6.5 Hz, 4H), 1.59 (t, J=3.5 Hz, 4H) ppm. ¹³C NMR (125 MHz, CDCl₃): δ=165.22, 163.41, 156.14, 145.02, 132.25, 122.64, 121.72, 115.38, 114.29, 69.28, 68.10, 50.13, 44.73, 29.05, 25.81 ppm. MS (+ESI): calcd for [C₃₈H₃₈O₁₀+H]+: m/z 655; found: m/z 655.

F) n=8

The synthesis was performed in the same manner as D), except that BPH8 was used instead of BPH4.

¹H NMR (500 MHZ, CDCl₃): δ=8.13 (d, J=9 Hz, 4H), 7.12 (d, J=9 Hz, 4H), 6.96 (d, J=8.5, 8H), 4.23 (dd, J=3 Hz, 1.5 Hz, 2H), 4.06 (t, J=6.5 Hz, 4H), 3.98 (dd, J=5.5 Hz, 2 Hz, 2H), 3.37 (m, 2H), 2.92 (t, J=5 Hz, 2H), 2.77 (m, 2H), 1.85 (t, J=7 Hz, 4H), 1.50 (m, 4H), 1.42 (m, 4H) ppm. 13C NMR (125 MHz, CDCl₃): 0=165.23, 163.23, 156.13, 145.03, 132.24, 122.64, 121.68, 115.37, 114.29 ppm. MS (+ESI): calcd for [C₄₀H₄₂O₁₀+H]⁺: m/z 683; found: m/z 683.

### Example 2: Synthesis of EIM-n (Bis (4-(((4-(oxiran-2-ylmethoxy) phenyl) imino) methyl) phenyl) alkanedioate)

Scheme 2 below shows the synthesis process of EIM-n.

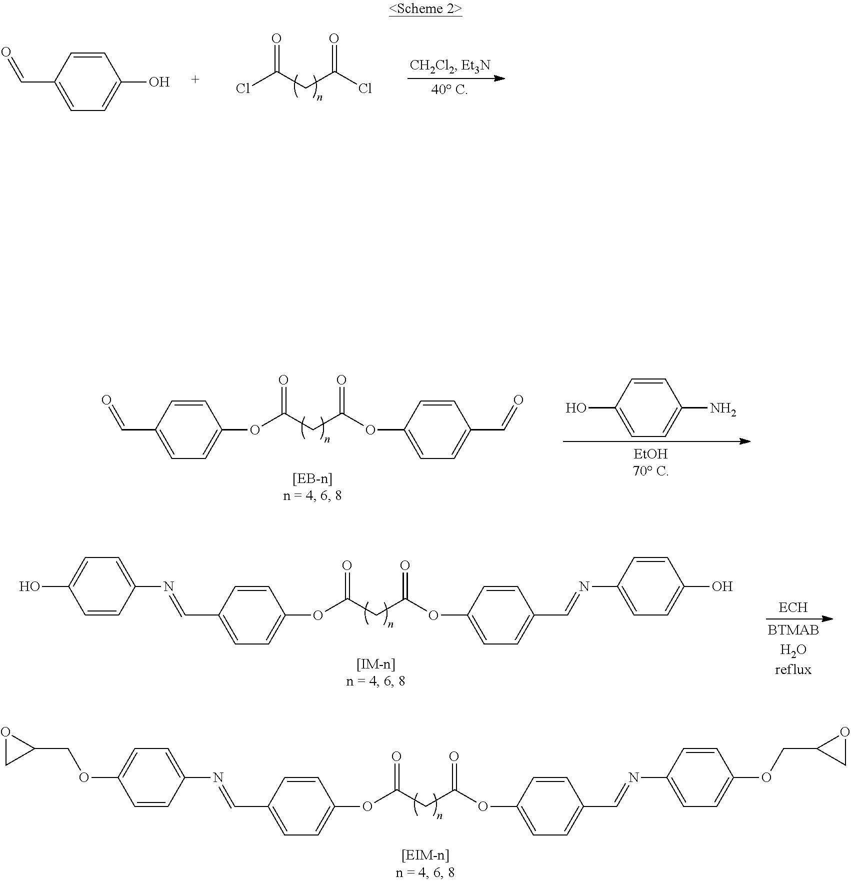

### \textcircled{1} Synthesis of bis (4-formylphenyl) alkanedioate (EB-n) of Formula (III) Below

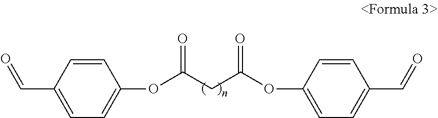

A) n=4

4-Hydroxylphenylbenzaldehyde (3.05 g, 25.0 mmol) was put into a two-necked flask, of which atmosphere was replaced with argon, and then 3.5 ml of triethylamine and 100 ml of anhydrous CH₂Cl₂ were added and stirred until a homogeneous solution was formed. Afterwards, 30 ml of anhydrous CH₂Cl₂ in which 1.82 ml (12.5 mmol) of hexanediol dichloride was dissolved was added dropwise and refluxed for 3 hours. The obtained solution was concentrated after extracting impurities three times with a saturated sodium bicarbonate solution, and purified through silica column chromatography using a chloroform:ethyl acetate solution in a volume ratio of 11:1 as an eluent. EB-4 was obtained in an amount of 3.69 g, and the yield was 85%.

¹H NMR (500 MHZ, CDCl₃): δ=10.00 (s, 2H (CHO—Ar)), 7.92 (d, J=8.6 Hz, 4ArH), 7.28 (d, J=8.6 Hz, 4ArH), 2,69 (t, J=7.4 Hz, 4H (—CO—CH₂—CH₂—)), 1.91 (q, J₁=3 Hz, J₂=4 Hz, 4H (—COCH₂—CH₂—)).

B) n=6

The synthesis was performed in the same manner as A), except that octanediol dichloride was used instead of hexanediol dichloride. The yield was 89%.

¹H NMR (500 MHZ, CDCl₃): δ=10 (s, 2H (CHO—Ar)), 7.92 (d, J=8.6 Hz, 4ArH), 7.27 (d, J=8.6 Hz, 4ArH), 2, 62 (t, J=7.4 Hz, 4H (—CO—CH₂—CH₂—CH₂)), 1.80 (quintet, J_{1=7.5} Hz, J₂=7.5 Hz, 4H (—COCH₂—CH₂—CH₂—)), 1.507 (quintet, J₁=4 Hz, J₂=4, 4H (—CO—CH₂—CH₂—CH₂—)).

C) n=8

The synthesis was performed in the same manner as A), except that decanediol dichloride was used instead of hexanediol dichloride. The yield was 86%.

¹H NMR (500 MHZ, CDCl₃): δ=10 (s, 2H (CHO—Ar)), 7.91 (d, J=9 Hz, 4ArH), 7.27 (d, J=8.6 Hz, 4ArH), 2,59 (t, J=7.4 Hz, 4H (—CO—CH₂—CH₂—CH₂—CH₂—)), 1.77 (quintet, J₁=7.5 Hz, J₂=7.5 Hz, 4H (—CO—CH₂—CH₂—CH₂—CH₂—)), 1.41-149 (m, 8H (—CO—CH₂—CH₂—CH₂—CH₂—)), 1.36-1.44 (m, 8H (—CO—CH₂—CH₂—CH₂—CH₂—)).

### \textcircled{2} Synthesis of bis (4-(((4-hydroxyphenyl) imino) methyl) phenyl) alkanedioate (IM-n) of Formula (IV) Below

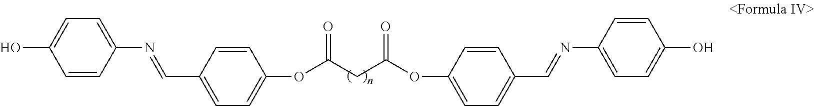

D) n=4

4-aminophenol (4.80 g, 44 mmol) was dissolved in 250 ml of anhydrous ethanol in a 1 L three-necked round flask, and EB-4 (22 mmol) and 250 ml of anhydrous ethanol were added to another 500 ml round flask to prepare a homogeneous solution. Afterwards, the two solutions were mixed, subjected to a reaction under reflux conditions for 30 minutes, and cooled to room temperature. The obtained crystals were washed three times with cold ethanol and then recovered. The recovered amount was 10.9 g, and the yield was 92%.

¹H NMR (500 MHZ, DMSO-d₆): δ=9.52 (s, 2H (OH—Ar), 8.61 (s, 2H (Ar—CH═N—Ar)), 7.94 (d, J=9 Hz, 4ArH), 7.27 (d, J=8.6 Hz, 4ArH), 7.20 (d, J=9 Hz, 4ArH), 6.80 (d, J=8.6 Hz, 4ArH), 2.70 (t, J=6 Hz, 4H (—CO—CH₂—CH₂—)), 1.74-1.82 (m, 4H (—CO—CH₂—CH₂—)).

E) n=6

The synthesis was performed in the same manner as D), except that EB-6 was used instead of EB-4. The yield was 95%.

¹H NMR (500 MHZ, DMSO-d₆): δ=9.55 (s, 2H (OH—Ar), 8.61 (s, 2H (Ar—CH═N—Ar)), 7.94 (d, J=9 Hz, 4ArH), 7.26 (d, J=8.6 Hz, 4ArH), 7.20 (d, J=8.6 Hz, 4ArH), 6.80 (d, J=9 Hz, 4ArH), 2.64 (t, J=7.5 Hz, 4H (—CO—CH₂—CH₂—CH₂—)), 1.65-1.73 (m, 4H (—CO—CH₂—CH₂—CH₂—)), 1.41-1.48 (m, 4H (—CO—CH₂—CH₂—CH₂—)).

F) n=8

The synthesis was performed in the same manner as D), except that EB-8 was used instead of EB-4. The yield was 94%.

¹H NMR (500 MHZ, DMSO-d₆): δ=9.55 (s, 2H (OH—Ar), 8.61 (s, 2H (Ar—CH═N—Ar)), 7.93 (d, J=9 Hz, 4ArH), 7.25 (d, J=8.6 Hz, 4ArH), 7.20 (d, J=8.6 Hz, 4ArH), 6.80 (d, J=8.6 Hz, 4ArH), 2.61 (t, J=7.5 Hz, 4H (—CO—CH₂—CH₂—CH₂—CH₂—)), 1.67 (quintet, J₁=7.3 Hz, J₂=7.3 Hz, 4H (—CO—CH₂—CH₂— CH₂—CH₂—)), 1.39-1.44 (m, 4H (—CO—CH₂—CH₂—CH₂—CH₂—)), 1.32-1.35 (m, 4H (—CO—CH₂—CH₂—CH₂—CH₂—)).

### \textcircled{3} Synthesis of bis (4-(((4-(oxiran-2-ylmethoxy) phenyl) imino) methyl) phenyl) alkanedioate (EIM-n) of Formula (V) Below

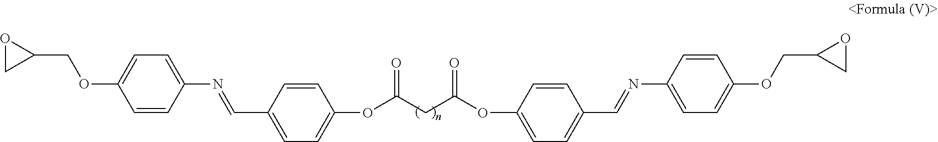

G) n=4

IM-4 (4.02 g, 7.5 mmol) and benzyl trimethylammonium bromide (0.4 g) were dissolved in 40 ml of anhydrous DMF, and 55 ml of epichlorohydrin was added and reacted at 90° C. for 1 hour. After cooling to room temperature, was the obtained solution precipitated in 300 ml of methanol, and the resulting white solid material was filtered and recovered. The obtained solid was washed several times with methanol and distilled water, and the dry weight was 2.04 g, and the yield was 42%.

¹H NMR (500 MHZ, CDCl₃): δ (ppm)=8.38 (s, 2H (Ar—CH═N—Ar)), 7.84 (d, J=8.6 Hz, 4ArH), 7.20 (d, J=9.0 Hz, 4ArH), 7.10 (d, J=9.0 Hz, 4ArH), 7.00 (d, J=8.6 Hz, 4ArH), 4.31 (dd, J₁=11.0 Hz, J₂=3.1 Hz, 2H), 4.02 (dd, J₁=11.0 Hz, J₂=5.8 Hz, 2H), 3.39 (ddt, J₁=5.8 Hz, J₂=4.1 Hz, J₃=2.9 Hz, 2H), 2.94 (dd, J₁=4.9 Hz, J₂=4.1 Hz, 2H), 2.79 (dd, J₁=4.9 Hz, J₂=2.6 Hz, 2H), 2.66 (q, J₁=6.1 Hz, J₂=4.2 Hz, 4H), 1.88-1.94 (m, 4H). ¹³C NMR (500 MHz, CDCl₃): δ (ppm): 171.86 (C═O), 161.11 (—C═N), 159.70, 148.51, 121.73, 114.80 (—O—Ar—N═) 149.85, 130.56, 129.60, 122.12 (ArO—), 68.82 (—CH₂—O), 49.96 (CH of oxirane ring), 44.61 (CH₂ of oxirane ring), 33.96 (—CO—CH₂—CH₂—), 24.29 (—CO—CH₂—CH₂—). MS (+ESI): calcd for [C₃₈H₃₆N₂O₈+2H]²⁺: m/z 650; found: m/z 650.

H) n=6

The synthesis was performed in the same manner as in G), except that IM-6 was used instead of IM-4. The yield was 44%.

¹H NMR (500 MHZ, CDCl₃): δ (ppm)=8.37 (s, 2H (Ar—CH═N—Ar)), 7.84 (d, J=9.0 Hz, 4ArH), 7.20 (d, J=9.0 Hz, 4ArH), 7.09 (d, J=9.0 Hz, 4ArH), 7.00 (d, J=8.6 Hz, 4ArH), 4.31 (dd, J₁=11.0, J₂=3.0 Hz, 2H), 4.01 (dd, J₁=11.0 Hz, J₂=5.7 Hz, 2H), 3.39 (ddt, J₁=5.7 Hz, J₂=4.2 Hz, J₃=2.9 Hz, 1H), 2.93 (dd, J₁=4.9 Hz, J₂=4.1 Hz, 1H), 2.79 (dd, J₁=4.9 Hz, J₂=2.6 Hz, 1H), 2.59 (t, J=7.4 Hz, 2H), 1.81 (dq, J₁=11.5 Hz, J₂=7.1 Hz, 2H), 1.51 (m, 4H). ¹³C NMR (500 MHZ, CDCl₃): δ (ppm): 172.25 (C═O), 161.09 (—C═N), 159.69, 148.57, 121.71, 114.79 (—O—Ar—N═), 149.73, 130.55, 129.55, 122.13 (ArO—), 68.80 (—CH₂—O), 49.95 (CH of oxirane ring), 44.58 (CH₂ of oxirane ring), 34.22 (—CO—CH₂—), 28.68 (—CO—CH₂—CH₂—), 24.68 (—CO—CH₂—CH₂—CH₂—). MS (+ESI): calcd for [C₄₀H₄₀N₂O₈+2H]²⁺: m/z 678; found: m/z 678.

I) n=8

The synthesis was performed in the same manner as in G), except that IM-8 was used instead of IM-4. The yield was 45%.

¹H NMR (500 MHZ, CDCl₃): δ (ppm)=8.37 (s, 2H (Ar—CH═N—Ar)), 7.84 (d, J=9.0 Hz, 4ArH), 7.19 (d, J=9.0 Hz, 4ArH), 7.09 (d, J=9.0 Hz, 4ArH), 7.00 (d, J=8.6 Hz, 4ArH), 4.31 (dd, J₁=11.1 Hz, J₂=3.1 Hz, 2H), 4.02 (dd, J₁=11.0 Hz, J₂=5.7 Hz, 2H), 3.39 (ddt, J₁=5.8 Hz, J₂=4.2 Hz, J₃=2.8 Hz, 2H), 2.93 (dd, J₁=4.9 Hz, J₂=4.1 Hz, 2H), 2.79 (dd, J₁=4.9 Hz, J₂=2.6 Hz, 2H), 2.57 (t, J=7.5 Hz, 4H), 1.77 (q, J=7.4 Hz, 4H), 1.38-1.48 (m, 8H). 13C NMR (500 MHZ, CDCl₃): δ (ppm): 172.36 (C═O), 161.08 (—C═N), 159.62, 148.60, 121.70, 114.79 (—O—Ar—N═), 149.74, 130.54, 129.59, 122.13 (ArO—), 68.80 (—CH₂—O), 49.95 (CH of oxirane ring), 44.59

(CH₂ of oxirane ring), 34.33 (—CO—CH₂—), 29.03 (—CO—CH₂—CH₂—), 28.99 (—CO—CH₂—CH₂—CH₂—), 24.87 (—CO—CH₂—CH₂— CH₂—CH₂—). MS (+ESI): calcd for [C₄₂H₄₄N₂O₈+2H]²⁺; m/z 706; found: m/z 706.

### Example 3: Synthesis of IBP-n (bis (4′-(oxiran-2-ylmethoxy)-[1,1′-biphenyl]-4-yl) alkanedioate)

Scheme 3 below shows the synthesis process of EBP-n.

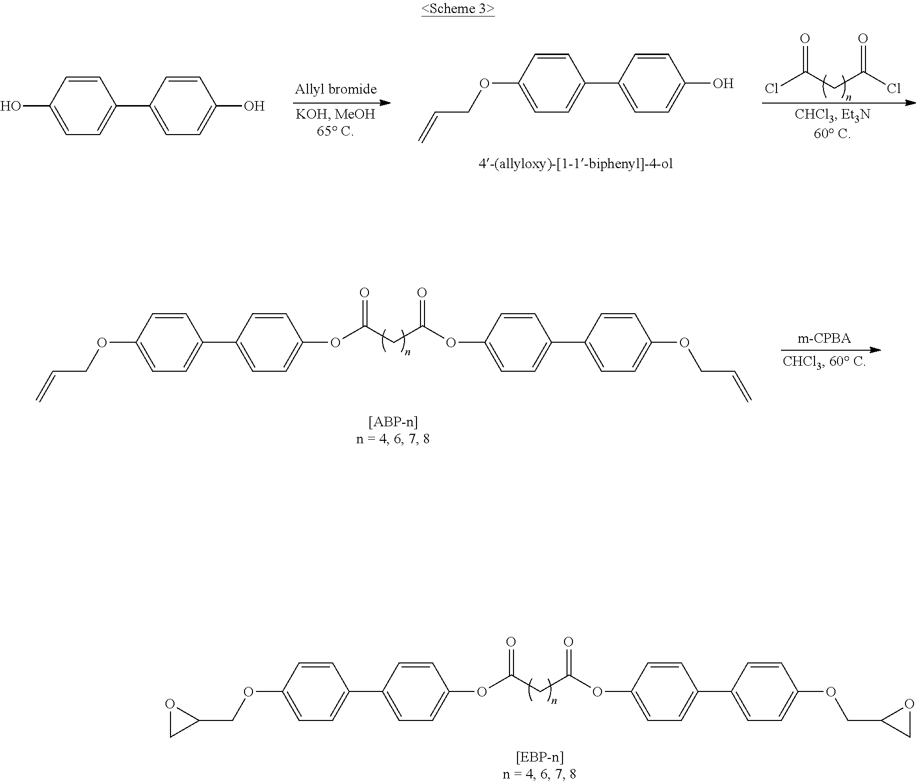

### \textcircled{1} Synthesis of bis (4′-(allyloxy)-[1,1′-biphenyl]-4-yl) alkanedioate (ABP-n) of Formula (VI) Below

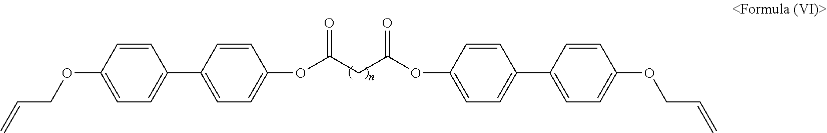

A) n=4

4′-(allyloxy)-[1,1′-biphenyl]-4-ol (3.00 g, 13.3 mmol) was put into a two-necked flask, of which atmosphere was replaced with argon, and then 5 ml of triethylamine and 100 ml of anhydrous CHCl₃ were added and stirred until a homogeneous solution was obtained. Afterwards, 0.97 ml (6.65 mmol) of hexanediol dichloride was added dropwise and refluxed at 60° C. for 5 hours. The obtained solution was concentrated after extracting impurities three times with a saturated sodium bicarbonate solution, and purified through silica column chromatography using a chloroform:ethyl acetate solution in a volume ratio of 19:1 as an eluent. ABP-4 was obtained in an amount of 1.6 g, and the yield was 43%.

¹H NMR (500 MHZ, CDCl₃): 0 (ppm)=7.53 (d, J=8.5 Hz, 4ArH), 7.48 (d, J=9.0 Hz, 4ArH), 7.14 (d, J=8.5 Hz, 4ArH), 6.98 (d, J=9 Hz, 4ArH), 6.08 (ddt, J₁=17.3, J₂=10.6 Hz, J₃=5.3 Hz, 2H, CH₂=CH—O—), 5.44 (dq, J₁=17.2 Hz, J₂=1.7 Hz, 2H, CH₂=CH—O—), 5.31 (dq, J₁=10.5, J₂=1.4 Hz, 2H, CH₂=CH—O—), 4.58 (dt, J₁=5.3, J₂=1.6 Hz, 4H, Ar—O—CH₂—), 2.63-2.70 (m, 4H, —CO—CH₂—), 1.89-1.95 (m, 4H, —COCH₂—CH₂—).

B) n=6

The synthesis was performed in the same manner as A), except that octanediol dichloride was used instead of hexanediol dichloride. The yield was 46%.

¹H NMR (500 MHZ, CDCl₃): δ (ppm)=7.53 (d, J=8.5 Hz, 4ArH), 7.48 (d, J=9.0 Hz, 4ArH), 7.12 (d, J=8.5 Hz, 4ArH), 6.98 (d, J=9 Hz, 4ArH), 6.08 (ddt, J₁=17.3, J₂=10.5 Hz, J₃=5.3 Hz, 2H, CH₂=CH—O—), 5.44 (dq, J₁=17.2 Hz, J₂=1.6 Hz, 2H, CH₂=CH-0-), 5.31 (dq, J₁=10.5, J₂=1.4 Hz, 2H, CH₂=CH—O—), 4.58 (dt, J₁=5.3, J₂=1.6 Hz, 4H, Ar—O—CH₂—), 2.60 (t, J=7.4 Hz, 4H, —CO—CH₂—), 1.79-1.84 (m, 4H, —CO—CH₂—CH₂—), 1.48-1.55 (m, 4H, —CO—CH₂—CH₂—CH₂—).

C) n=7

The synthesis was performed in the same manner as A), except that nonanediol dichloride was used instead of hexanediol dichloride. The yield was 81%.

¹H NMR (500 MHZ, CDCl₃): δ (ppm)=7.53 (d, J=8.5 Hz, 4ArH), 7.47 (d, J=8.5 Hz, 4ArH), 7.12 (d, J=8.5 Hz, 4ArH), 6.98 (d, J=8.5 Hz, 4ArH), 6.08 (ddt, J₁=17.3, J₂=10.5 Hz, J₃=5.3 Hz, 2H, CH₂=CH—O—), 5.44 (dq, J₁=17.2 Hz, J₂=1.6 Hz, 2H, CH₂=CH-0-), 5.31 (dq, J₁=10.5, J₂=1.4 Hz, 2H, CH₂=CH—O—), 4.58 (dt, J₁=5.3, J₂=1.5 Hz, 4H, Ar—O—CH₂—), 2.59 (t, J=7.4 Hz, 4H, —CO—CH₂—), 1.79 (q, J=7.3 Hz, 4H, —CO—CH₂—CH₂—), 1.44-1.53 (m, 6H, —CO—CH₂—CH₂—CH₂—CH₂—).

D) n=8

The synthesis was performed in the same manner as A), except that decanediol dichloride was used instead of hexanediol dichloride. The yield was 44%.

¹H NMR (500 MHZ, CDCl₃): δ (ppm)=7.53 (d, J=8.5 Hz, 4ArH), 7.48 (d, J=8.5 Hz, 4ArH), 7.12 (d, J=9.0 Hz, 4ArH), 6.98 (d, J=8.5 Hz, 4ArH), 6.08 (ddt, J₁=17.2, J₂=10.5 Hz, J₃=5.3 Hz, 2H, CH₂=CH—O—), 5.44 (dq, J₁=17.3 Hz, J₂=1.7 Hz, 2H, CH₂=CH-0-), 5.31 (dq, J₁=10.5, J₂=1.4 Hz, 2H, CH₂=CH—O—), 4.58 (dt, J₁=5.3, J₂=1.5 Hz, 4H, Ar—O—CH₂—), 2.58 (t, J=7.5 Hz, 4H, —CO—CH₂—), 1.78 (q, J=7.4 Hz, 4H, —CO—CH₂—CH₂—), 1.38-1.46 (m, 8H, —CO—CH₂—CH₂—CH₂—CH₂—).

### \textcircled{2} Synthesis of bis (4′-(oxiran-2-ylmethoxy)-[1,1′-biphenyl]-4-yl) alkanedioate (EBP-n) of Formula (VII) Below

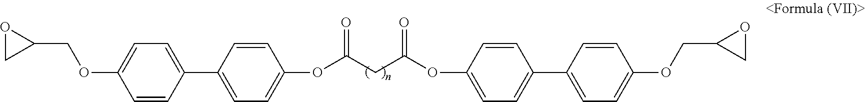

E) n=4

ABP-4 (2.0 g, 3.56 mmol) was added to a 500 ml three-necked round flask, and 100 ml of anhydrous CHCl₃ was added and stirred to prepare a homogeneous solution. Then, 4.92 g of m-CPBA was added and refluxed for 10 hours. The organic phase was washed three times each with saturated aqueous NaHSO₃ and NaHCO₃solutions, then concentrated, and the obtained solid was purified through silica column chromatography using a chloroform:ethyl acetate solution in a volume ratio of 13:1 as an eluent. EBP-4 was obtained in an amount of 1.28 g, and the yield was 60%.

¹H NMR (500 MHZ, CDCl₃): δ (ppm)=7.53 (d, J=8.5 Hz, 4ArH), 7.49 (d, J=9 Hz, 4ArH), 7.14 (d, J=9.0 Hz, 4ArH), 6.99 (d, J=8.5 Hz, 4ArH), 4.26 (dd, J₁=11.0 Hz, J₂=3.2 Hz, 2H, ArO—CH₂), 4.01 (dd, J₁=11.0 Hz, J₂=5.6 Hz, 2H, ArO—CH₂), 3.38 (ddt, J₁=5.8, J₂=4.1 Hz, J₃=2.9 Hz, 2H, CH of oxirane ring), 2.93 (dd, J₁=4.9 Hz, J₂=4.1 Hz, 2H, CH₂ of oxirane ring), 2.78 (dd, J₁=4.9 Hz, J₂=2.7 Hz, 2H, CH₂ of oxirane ring), 2.63-2.71 (m, 4H, —CO—CH₂—), 1.92 (q, J=3.7 Hz, 4H, —CO—CH₂—CH₂—).

F) n=6

The synthesis was performed in the same manner as E), except that ABP-6 was used instead of ABP-4. The yield was 62%.

¹H NMR (500 MHZ, CDCl₃): δ (ppm)=7.53 (d, J=9.0 Hz, 4ArH), 7.48 (d, J=8.5 Hz, 4ArH), 7.12 (d, J=8.5 Hz, 4ArH), 6.99 (d, J=8.5 Hz, 4ArH), 4.26 (dd, J₁=11.0 Hz, J₂=3.2 Hz, 2H, ArO—CH₂), 4.01 (dd, J₁=11.0 Hz, J₂=5.6 Hz, 2H, ArO—CH₂), 3.38 (ddt, J₁=5.8, J₂=3.8 Hz, J₃=2.7 Hz, 2H, CH of oxirane ring), 2.93 (dd, J₁=4.9 Hz, J₂=4.1 Hz, 2H, CH₂ of oxirane ring), 2.78 (dd, J₁=4.9 Hz, J₂=2.6 Hz, 2H, CH₂ of oxirane ring), 2.60 (t, J=7.5 Hz, 4H, —CO—CH₂—), 1.48-1.55 (m, 8H, —CO—CH₂—CH₂— CH₂—).

### Test Example 1—Phase Transition Characteristics

The phase transition phenomenon was investigated by using PerkinElmer's DSC4000 differential scanning calorimeter and Olympus' BX53-P polarizing microscope.

Tables 1 to 3 are the DSC measurement results of EBH-n, EIM-n, and EBP-n, respectively. Referring to these, they showed the phase transition phenomenon of typical thermotropic liquid crystals in both heating and cooling, and when the chain length was longer, the transition temperature was lower, except for EBP-7.

TABLE 1

| | Heating | | | Cooling | |
|---|---|---|---|---|---|
| Sample | K → LC1 | LC1 → LC1* | LC1 → I | I → LC | LC → K |
| EBH-4 | 151° C. | 178° C. | 212° C. | 211° C. | 128° C. |
| | ΔH = 37 J/g | ΔH = 6 J/g | ΔH = 9 J/g | ΔH = −10 J/g | ΔH = −46 J/g |
| EBH-6 | 147° C. | n/d | 192° C. | 192° C. | 112° C. |
| | ΔH = 31 J/g | | ΔH = 8 J/g | ΔH = −8 J/g | ΔH = −26 J/g |
| EBH-8 | 117° C. | n/d | 169° C. | 171° C. | 95° C. |
| | ΔH = 40 J/g | | ΔH = 7 J/g | ΔH = −9 J/g | ΔH = −40 J/g |

TABLE 2

| | Heating | | | | Cooling | | | | |
|---|---|---|---|---|---|---|---|---|---|
| Sample | K → LC | ΔH | LC → I | ΔH | I → LC | ΔH | LC → K | ΔH | |
| EIM-4 | 124° C. | 33.7 J/g | 202° C. | 7.8 J/g | 212° C. | −8.1 J/g | 116° C. | −31.5 | J/g |
| EIM-6 | 118° C. | 32.3 J/g | 186° C. | 4.8 J/g | 193° C. | −5.0 J/g | 106° C. | −29.9 | J/g |
| EIM-8 | 119° C. | 33.0 J/g | 180° C. | 7.2 J/g | 184° C. | −7.7 J/g | 110° C. | 29.5 | J/g |

TABLE 3

| | |
|---|---|
| Heating | Cooling |

| Sample | K → LC | LC → I | I → LC | LC → K |
|---|---|---|---|---|
| EBP-4 | 204° C. | 234° C. | 232° C. | 193° C. |
| EBP-6 | 184° C. | 209° C. | 179° C. | 208° C. |
| EBP-7 | 164° C. | 168° C. | 178° C. | 168° C. |
| EBP-8 | 179° C. | 191° C. | 191° C. | 173° C. |

### Preparation Example 1—Preparation of EBH-n Cured Product of Example 1

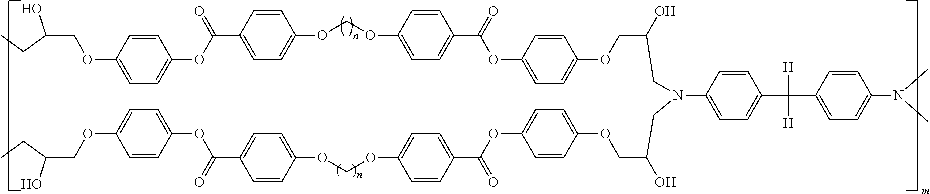

The molar mixing ratio of an epoxy resin and the curing agent 4,4′-diaminodiphenylmethane was set to 2:1 so that the epoxide and amine equivalent weights were the same, and the two materials in the solid state at room temperature were crushed and mixed to prepare a cured product by hot press molding. The curing temperature was 130° C., and the curing time was 1 hour.

### Test Example 2—Physical Properties of EBH-n Cured Product

The glass transition temperature of the cured product was measured with a differential scanning calorimeter, the decomposition temperature was measured with TA's Q500 thermogravimetric analyzer, and the thermal conductivity was measured with Hotdisk's TPS 2500S thermal conductivity meter, and the measurement results are shown in Table 4. The glass transition temperature of the cured product was in a range from 79 to 127° C. and tended to decrease as the chain length increased. The 5% weight loss temperature was in a range from 329 to 337° C., and the 10% weight loss temperature was in a range from 339 to 346° C. Although there was no significant difference, it was slightly increased as the chain length was increased. The thermal conductivity was confirmed in a range from 0.38 to 0.48 W/m·K. Although there was no particular tendency, from the liquid crystal temperature range of the monomers, the highest thermal conductivity was confirmed in EBH-8, which has a liquid crystalline phase at the curing temperature, and so it was confirmed that there was a correlation between the formation of a liquid crystalline phase and the thermal conductivity values.

TABLE 4

| | | | | Thermal |
|---|---|---|---|---|
| | T | T_{d, 5%} | T_{d, 10%} | conductivity |
| Sample | [DSC^{g}, ° C.] | [° C.] | [° C.] | [W/m · K] |
| EBH-4/DDM | 127 | 329 | 339 | 0.44 |
| EBH-6/DDM | 98 | 334 | 343 | 0.38 |
| EBH-8/DDM | 79 | 337 | 346 | 0.48 |

### Preparation Example 2—Preparation of EIM-n Cured Product of Example 2

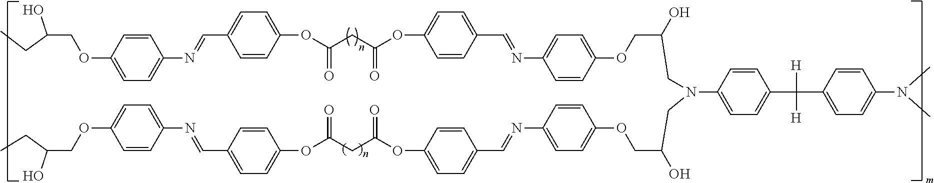

The molar mixing ratio of an epoxy resin and 4,4′-diaminodiphenylmethane was set to 2:1 so that the epoxide and amine equivalent weights were the same, and the two materials in the solid state at room temperature were crushed and mixed to prepare a cured product by hot press molding. The curing temperature was 130° C., and the curing time was 1 hour.

### Test Example 3—Physical Properties of EIM-n Cured Product

The glass transition temperature of the cured product was measured with a differential scanning calorimeter, the decomposition temperature was measured with a thermogravimetric analyzer, and the thermal conductivity was measured with a thermal conductivity meter, and the measurement results are shown in Table 5. The glass transition temperature of the cured product was in a range from 90 to 123° C. and tended to decrease as the chain length increased. The 5% weight loss temperature was in a range from 290 to 307° C., and the 10% weight loss temperature was in a range from 333 to 355° C. Although there was no significant difference, it was slightly increased as the chain length was increased. The thermal conductivity was confirmed in a range from 0.33 to 0.53 W/m·K, and it was confirmed that the thermal conductivity decreased as the chain length increased. The highest thermal conductivity was confirmed in EIM-8, which has the lowest liquid crystal transition temperature.

TABLE 5

| | | | | Thermal |
|---|---|---|---|---|
| | T | T_{d, 5%} | T_{d, 10%} | conductivity |
| Sample | [DSC^{g}, ° C.] | [° C.] | [° C.] | [W/m · K] |
| EIM-4/DDM | 123 | 290 | 333 | 0.33 |
| EIM-6/DDM | 97 | 307 | 352 | 0.42 |
| EIM-8/DDM | 90 | 307 | 355 | 0.53 |

Preparation Example 3—Preparation of EBP-n Cured Product of Example 3

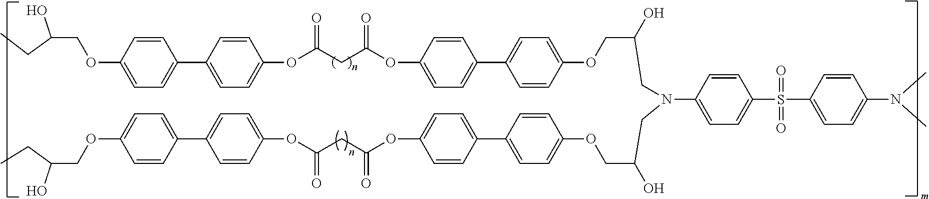

The molar mixing ratio of an epoxy resin and 4,4′-diaminodiphenylsulfone was set to 2:1 so that the epoxide and amine equivalent weights were the same, and the two materials in the solid state at room temperature were crushed and mixed to prepare a cured product by hot press molding. The curing temperature was 190° C., and the curing time was 2 hours.

### Test Example 3—Physical Properties of EBP-n Cured Product

The glass transition temperature of the cured product was measured with a differential scanning calorimeter, the decomposition temperature was measured with a thermogravimetric analyzer, and the thermal conductivity was measured with a thermal conductivity meter, and the measurement results are shown in Table 6. The glass transition temperature of the cured product was in a range from 125 to 150° C. and tended to decrease as the chain length increased, except for EBP-7. The 5% weight loss temperature was in a range from 349 to 361° C., and the 10% weight loss temperature was in a range from 368 to 380° C., and it was confirmed that they had a considerably high level of thermal stability due to the effect of the rigid biphenol structure. The thermal conductivity was confirmed in a range from 0.40 to 0.55 W/m·K, and the highest thermal conductivity was obtained from EBP-6, in which the liquid crystalline expression temperature was best matched with the curing temperature.

TABLE 6

| | | | | Thermal |
|---|---|---|---|---|
| | T | T_{d, 5%} | T_{d, 10%} | conductivity |
| Sample | [DSC^{g}, ° C.] | [° C.] | [° C.] | [W/m · K] |
| EBP-4/DDS | 150 | 351 | 372 | 0.47 |
| EBP-6/DDS | 136 | 353 | 370 | 0.55 |
| EBP-7/DDS | 140 | 361 | 380 | 0.40 |
| EBP-8/DDS | 125 | 349 | 368 | 0.43 |

### Comparative Example 1

Preparation of diglycidyl ether cured product of bisphenol A As diglycidyl ether of bisphenol A, which is a bifunctional epoxy resin, YD-128 from Kukdo Chemical was purchased and used. The epoxy equivalent weight (EEW) was 187 g/eq. The two materials were mixed at room temperature with the same amine equivalent weight and heated in a general-purpose convection oven to prepare a specimen. The curing temperature of the system using DDM as a curing agent was 130° C., and the curing time was 1 hour. The curing temperature of the system using DDS as a curing agent was 190° C., and the curing time was 2 hours.

### Test Example 5—Physical Properties of Diglycidyl Ether Cured Product of Bisphenol A

The glass transition temperature of the two types of diglycidyl ether cured products of bisphenol A was investigated by using a differential scanning calorimeter, the decomposition temperature was investigated by using a thermogravimetric analyzer, and the thermal conductivity was investigated by using a thermal conductivity meter, and the measurement results are shown in Table 7. The transition glass temperature of the 4,4′-diaminodiphenylmethane cured product was 144° C., the 10% weight loss temperature was 364° C., the thermal conductivity was 0.24 W/m·K. The glass transition temperature of the DDS cured product was 174° C., the 10% weight loss temperature was 404° C., and the thermal conductivity was 0.27 W/m·K. Compared to Examples 1 to 3, it was confirmed that the cured product of Comparative Example 1 had a slightly higher glass transition temperature and exhibited no significant difference in the decomposition temperature, but the thermal conductivity was considerably lower.

TABLE 7

| | | | | Thermal |
|---|---|---|---|---|
| | T | T_{d, 5%} | T_{d, 10%} | conductivity |
| Sample | [DSC^{g}, ° C.] | [° C.] | [° C.] | [W/m · K] |
| Diglycidylether cured | 144 | 355 | 364 | 0.24 |
| products of bisphenol | | | | |
| A/4,4′-diaminodiphenylmethane | | | | |
| Diglycidylether cured | 174 | 394 | 404 | 0.27 |
| products of bisphenol | | | | |
| A/4,4′-diaminodiphenyl | | | | |
| sulfone | | | | |

### Comparative Example 2

### Preparation of 4,4′-diglycidyloxybiphenyl Cured Product

Using biphenol as a starting material, 4,4′-diglycidyloxybiphenyl epoxy was synthesized through a reaction with epichlorohydrin in the presence of a base. It was prepared in the form of an oligomer by adjusting the base equivalent, and the epoxy equivalent was 190 g/eq. The two materials in the solid state at room temperature were crushed and mixed to prepare a cured product of the synthesized 4,4′-diglycidyloxybiphenyl epoxy by hot press molding with the same amine equivalent weights. The curing temperature of the system using 4,4′-diaminodiphenylmethane as a curing agent was 130° C., and the curing time was 1 hour. The curing temperature of the system using 4,4′-diaminodiphenyl sulfone as a curing agent was 190° C., and the curing time was 2 hours.

### Test Example 6—Physical Properties of 4,4′-diglycidyloxybiphenyl Cured Product

The glass transition temperature of the two types of 4,4′-diglycidyloxybiphenyl cured product was investigated by using a differential scanning calorimeter, the decomposition temperature was investigated by using a thermogravimetric analyzer, and the thermal conductivity was investigated by using a thermal conductivity meter, and the measurement results are shown in Table 8. The glass transition temperature of the DDM cured product was 160° C., the 10% weight loss temperature was 356° C., the thermal conductivity was 0.30 W/m·K. The glass transition temperature of the DDS cured product was 208° C., the 10% weight loss temperature was 393° C., and the thermal conductivity was 0.34 W/m·K. Compared to Examples 1 to 3, it was confirmed that the cured product of Comparative Example 1 had a higher glass transition temperature due to the rigid structure and exhibited a slightly higher decomposition temperature, but the thermal conductivity was considerably different.

TABLE 8

| | | | | Thermal |
|---|---|---|---|---|
| | T | T_{d, 5%} | T_{d, 10%} | conductivity |
| Sample | [DSC^{g}, ° C.] | [° C.] | [° C.] | [W/m · K] |
| BP/DDM | 160 | 342 | 356 | 0.30 |
| BP/DDS | 208 | 375 | 393 | 0.34 |

From the description above, those skilled in the art to which the present invention pertains will understand that the present invention can be implemented in other specific forms without changing its technical idea or essential features. In this regard, the examples described above should be understood in all respects as illustrative and not restrictive.

1. A multifunctional epoxy compound represented by Formula (I) below: 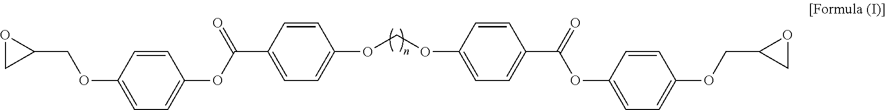
[In the Formula, n is an integer in a range from 1 to 30.]

2. A multifunctional epoxy compound represented by Formula (II) below: 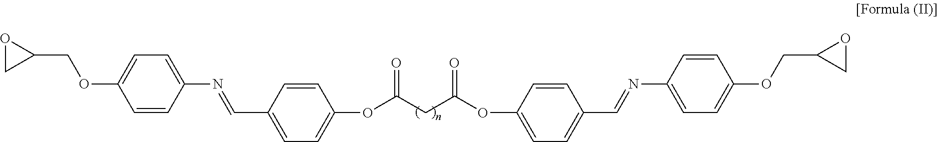
[In the Formula, n is an integer in a range from 1 to 30.]

3. A multifunctional epoxy compound represented by Formula (III) below: 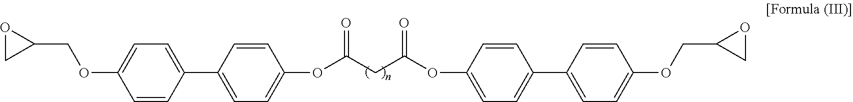
[In the Formula, n is an integer in a range from 1 to 30.]

4. A cured epoxy resin product obtained by reacting a compound according to claim 1 with a curing agent.

5. A cured epoxy resin product obtained by reacting a compound according to claim 2 with a curing agent.

6. A cured epoxy resin product obtained by reacting a compound according to claim 3 with a curing agent.

7. The cured epoxy resin product according to claim 4, wherein the cured product is represented by Formula (IV) below: 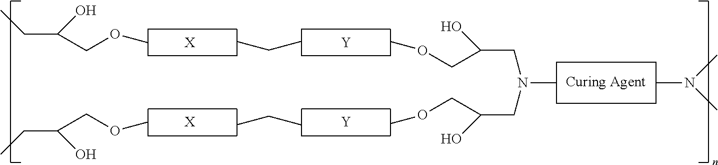
[In the Formula, X and Y may be the same or different, and are selected from the compounds represented by Formulas (I) to (III).]

8. The cured epoxy resin product according to claim 4, wherein the curing agent is selected from the group consisting of 4,4′-diaminodiphenylmethane (DDM), diaminodiphenylsulfone (DDS), m-phenylenediamine (mPDA), and dicyandiamide (DICY).

9. The cured epoxy resin product according to claim 4, wherein the cured product is used in substrates, compounds, adhesives, pads, heat spreads, and heat sinks.

10. The cured epoxy resin product according to claim 5, wherein the cured product is represented by Formula (IV) below: 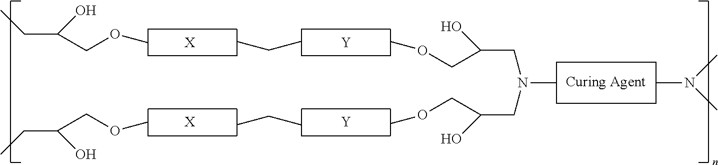
[In the Formula, X and Y may be the same or different, and are selected from the compounds represented by Formulas (I) to (III).]

11. The cured epoxy resin product according to claim 5, wherein the curing agent is selected from the group consisting of 4,4′-diaminodiphenylmethane (DDM), diaminodiphenylsulfone (DDS), m-phenylenediamine (mPDA), and dicyandiamide (DICY).

12. The cured epoxy resin product according to claim 5, wherein the cured product is used in substrates, compounds, adhesives, pads, heat spreads, and heat sinks.

13. The cured epoxy resin product according to claim 6, wherein the cured product is represented by Formula (IV) below: 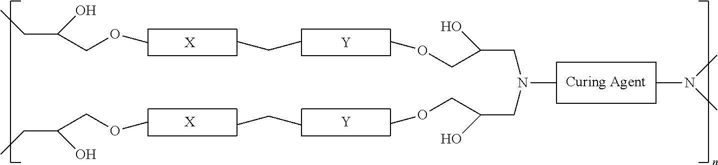
[In the Formula, X and Y may be the same or different, and are selected from the compounds represented by Formulas (I) to (III).]

14. The cured epoxy resin product according to claim 6, wherein the curing agent is selected from the group consisting of 4,4′-diaminodiphenylmethane (DDM), diaminodiphenylsulfone (DDS), m-phenylenediamine (mPDA), and dicyandiamide (DICY).

15. The cured epoxy resin product according to claim 6, wherein the cured product is used in substrates, compounds, adhesives, pads, heat spreads, and heat sinks.
